(19) United States
(12) Patent Application Publication
(10) Pub. No.: US 2005/0038240 A1
(43) Pub. Date: Feb. 17, 2005

(54) Processes for preparing 4'-azido-nucleoside derivatives

(75) Inventors: Terrence Joseph Connolly, Redwood City, CA (US); Kieran Durkin, Folsom, CA (US); Keshab Sarma, Sunnyvale, CA (US); Jiang Zhu, Cupertino, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 10/871,917

(22) Filed: Jun. 18, 2004

Related U.S. Application Data
(60) Provisional application No. 60/479,796, filed on Jun. 19, 2003.

(51) Int. Cl. C07H 19/067; C07D 45/04

(52) U.S. Cl. 536/28.51; 544/310

(57) ABSTRACT

A process for the preparation of a 4′-azido-2′,3′,5′-triacyl-nucleoside compound (I; B=B1; R¹ is R^{1a}CO— and R² is R^{2a}CO—) or a 4′-azidonucleoside compounds (I; B is B1 or B2 and R¹ and R² are hydrogen and acid addition salts thereof) wherein R^{1a} and R^{2a} are independently C₁₋₁₀ alkyl or phenyl optionally substituted with 1 to 3 substituents selected from the group consisting of alkyl, alkoxy, halogen, nitro or cyano and R³ is selected from the group consisting of hydrogen, C₁₋₆ alkyl, C₁₋₃ haloalkyl and halogen, comprising contacting a 5′-iodo compound II with a peracid, R^{2a}C(O)OOH, an acid R^{2a}C(O)OH and a phase transfer catalyst and interconverting a uridine B1 to a cytosine B2. The present process provides the 4′-azidonucleosides safely and selectively in high purity with increased efficiency.

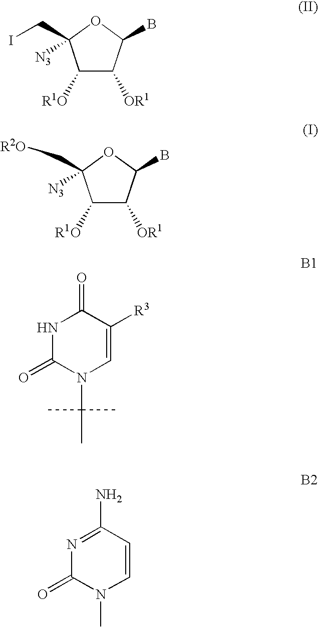

### CROSS REFERENCE TO PRIOR APPLICATION

This application claims benefit under Title 35 U.S.C. 119(e) of U.S. Provisional Application No. 60/479,796, filed Jun. 19, 2003, which is hereby incorporated by reference in its entirety.

### FIELD OF THE INVENTION

The invention relates to a novel process to prepare 4′-azidonucleoside derivatives that are useful for treating virus mediated diseases. More specifically the invention relates to a process for preparing 4-amino-1-((2R,3R,4S,5R)-5-azido-3,4-dihydroxy-5-hydroxymethyl-tetrahydro-furan-2-yl)-pyrimidin-2-one and pharmaceutically acceptable acid addition salts thereof and more particularly the hemisulfate salt.

### BACKGROUND OF THE INVENTION

The invention relates to processes for the preparation of azido substituted nucleoside derivatives useful for treating viral-mediated diseases. In particular, the invention is concerned with processes to prepare 4-azido pyrimidine nucleoside derivatives which are useful inhibitors of Hepatitis C Virus (HCV) RNA replication.

Hepatitis C virus (HCV) is responsible for a large proportion of the chronic liver disease worldwide and accounts for 70% of cases of chronic hepatitis in industrialized countries. The global proportion of hepatitis C is estimated to average 3% (ranging from 0.1% to 5.0%) and there are an estimated 170 million chronic carriers throughout the world. There is a continuing need for effective therapeutic agents against HCV and processes for the manufacture of such agents.

J. G. Moffatt (Chemical Transformations of the Sugar Moiety of Nucleosides, in Nucleoside Analogues, R. T. Walker, E. De Clercq and F. Eckstein, eds., Plenum Publishing Corp., New York, 1979, p. 144) describe the preparation of 4′-azidocytidine (VIIIb) by electrophilic addition of iodine azide to N-[1-((2R,3R,4S)-3,4-dihydroxy-5-methylene-tetrahydro-furan-2-yl)-2-oxo-1,2-dihydro-pyrimidin-4-yl]-benzamide (X).

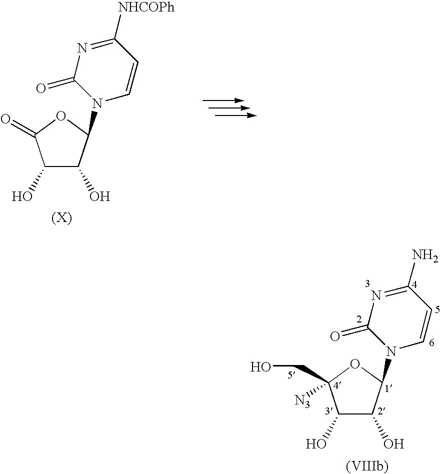

Maag et al. (J. Med. Chem. 1992 35:1440-1451) disclose the preparation of 4′-azido nucleosides by addition of iodine azide to 5-methylene-tetrahydro-furan-2-yl nucleosides XI wherein B is 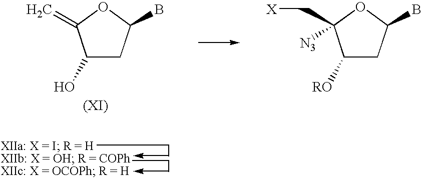
thymine, uracil, adenine or guanosine. Maag et al. (supra) further disclose that although displacement of the 5′-iodo is retarded by electron-withdrawing groups at the 4′-position, contacting (XIIa) with perbenzoic acid derivatives oxidize the iodide to a hypervalent state which is then displaced to afford a mixture of products including XIIb and XIIc. The reaction was suggested to proceed via a 3′-5′-cyclic benzoxonium ion. The criticality of the proximal 3′-acyloxy moiety was apparent when the reaction failed with a 3′-deoxy-nucleoside or a nucleoside unesterified at the 3′-position.

WO 02/100415 (R. Devos et al.) discloses 4′-substituted nucleoside derivatives which inhibit viral DNA polymerase. 4′-Azidonucleosides were prepared by the method described by Maag et al. (supra). The acetonide of uridine IVd was iodinated using a mixture of TPP, iodine and pyridine to afford the corresponding 5′-iodo derivative IVe. The acetonide protecting group was removed by treatment with an acid, for instance acetic acid, as described by J. P. Verheyden et al. (J. Org. Chem., 1970, 35(7): 2319) to afford nucleosides of formula IVf which were dehydroiodinated with sodium methoxide to afford Va. Treatment of Va with a mixture of iodine chloride and sodium azide in DMF afforded iodoazides nucleosides of formula IIa. After the hydroxy groups of IIa were protected by treatment of with benzoyl chloride in pyridine to afford diacyl nucleosides of formula IId, the diesters were converted into the 5′-benzoyl nucleosides of formula IIIb by treatment with MCPBA in dichloromethane. The uridine IIIb was converted to cytidine by protecting the 3′-hydroxy with Ac₂O and pyridine and utilizing the method described by A. D. Borthwick et al., (J. Med. Chem. 1990, 33(1):179; see also K. J. Divakar and C. B. Reese J. Chem Soc., Perkin Trans. I 1982 1171-1176). Thus IIIc was treated with 4-chlorophenyl dichlorophosphate and 1,2,4-triazole to give 4-triazolyl nucleosides of formula XIII, which was displaced with aqueous ammonia giving 4′-substituted cytidines of formula VIII.

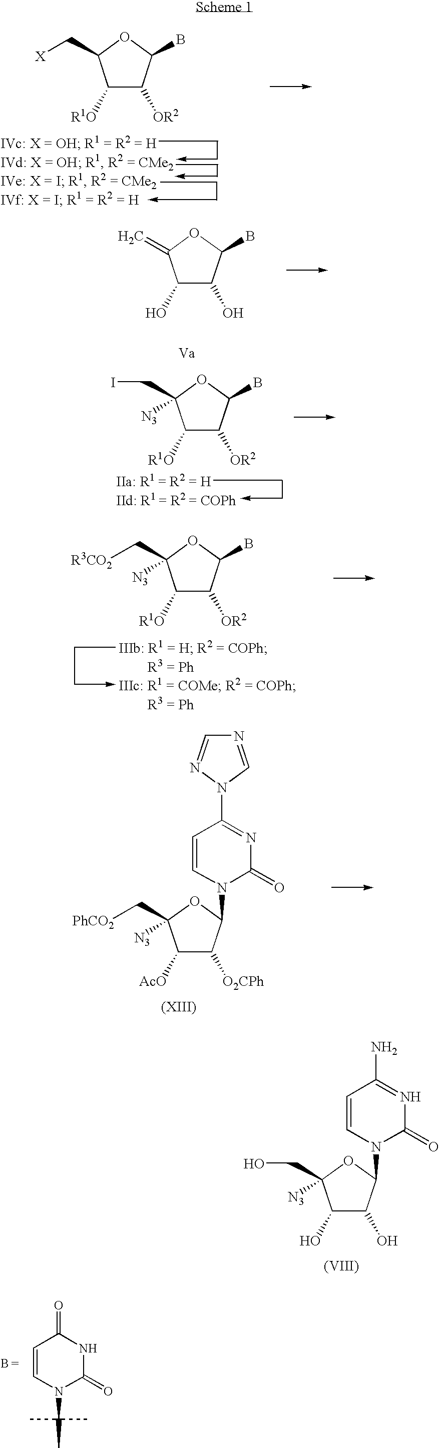

### SUMMARY OF THE INVENTION

The present invention further relates to processes to prepare 4′-azido-uridine derivatives (I) or 4′-azido-cytidine (VIII) or acid addition salts thereof.

### DETAILED DESCRIPTION OF THE INVENTION

4′-Azidocytidine has now been found to possess excellent activity against HCV polymerase. There is a need for new efficient processes to prepare VIII, and acid addition salts thereof, that minimizes process steps and reduces dependence on inefficient protecting strategies. Further the process steps must be compatible with a thermolabile azide moiety. The present invention relates to novel processes to prepare 4′-azidonucleoside compounds and 4′-azido-2′,3′,5′-tri-acylnucleoside compounds.

In one embodiment of the present invention there is provided a process for the preparation of a 4′- 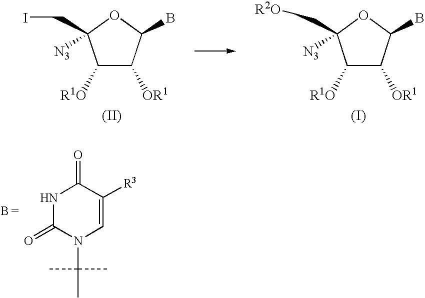
azido-2′,3′,5′-triacyl-nucleoside compound according to formula I, wherein R¹ is R^{1a}CO—, R² is R^{2a}CO—, R^{1a} and R^{2a} are independently C₁₋₁₀ alkyl or phenyl optionally substituted with 1 to 3 substituents selected from the group consisting of alkyl, alkoxy, halogen, nitro or cyano; R³ is selected from the group consisting of hydrogen, C₁₋₆ alkyl, C₁₋₃ haloalkyl and halogen, said process comprising contacting a solution of a 5′-iodomethyl-2′,3′-diacyl nucleoside compound II in a suitable solvent with a carboxylic peracid, R^{2a}C(O)OOH, a carboxylic acid R^{2a}C(O)OH and optionally a phase transfer catalyst.

Surprisingly the displacement of the iodo-dibenzoate (II) by activation of the leaving group with an oxidizing agent (MCPBA), and displacement with an external nucleophile (MCBA) in a suitable solvent containing a phase-transfer catalyst resulted in significantly higher yields of a triester then the displacement reaction described by Maag et al. (supra). The reaction directly affords a triester and eliminates the need for a separate reaction step to esterify the 3′-hydroxyl group formed in the absence of an external nucleophile. To activate the leaving group the iodide is oxidized to a hypervalent state with a carboxylic peracid. The peracid activation is necessitated by the presence of an electron-withdrawing group at the 4′-position. Previous studies have shown that the labile hypervalent iodide was displaced by an intramolecular transfer of the acyl moiety on the 3′-hydroxyl to the 5′-hydroxyl. The present reaction, in contrast, proceeds via an intermolecular displacement by carboxylic acid salt (e.g., potassium m-chlorobenzoate, sodium benzoate, sodium acetate) rather than intramolecular transfer of the 3-acyl moiety. The reaction can be carried out in a variety of solvents. Any organic solvent which is sufficient polar to dissolve the salt of the nucleophilic carboxylic acid is acceptable; however, a two-phase media including a buffered aqueous solution to provide nucleophilic carboxylic acid salt, a phase-transfer catalyst and a non-polar organic solvent is especially suitable. A two-phase media comprising water and dichloromethane is preferred due to non-flammability of the organic solvent. Suitable buffers afford a pH from about 2 to about 10 and include, but are not limited to potassium phosphate, potassium hydrogen phosphate, dihydrogen potassium, sodium carbonate and sodium bicarbonate. The present process allows for the introduction a specific 5-acyloxy moiety in good yield and high purity.

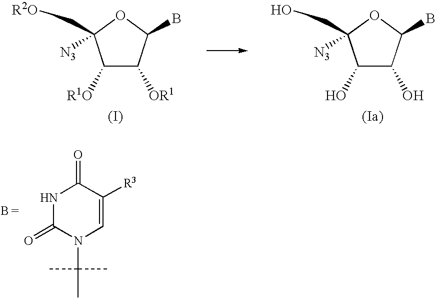

In another embodiment of the present invention there is provided a process for the preparation of a 4′-azido-nucleoside compound Ia wherein R³ is as defined hereinabove, said process comprising (i) contacting a solution of a 5′-iodomethyl-2′,3′-diacyl nucleoside compound II in a suitable solvent with a carboxylic peracid, R^{2a}C(O)OOH, a carboxylic acid R^{2a}C(O)OH and optionally a phase transfer catalyst to afford I wherein R′, R² and R³ are as described hereinabove; and (ii) contacting the resulting 4′-azido-2′,3′,5′-triacyl-nucleoside compound I with a base to afford the pyrimidine nucleoside compound according to formula Ia affording a novel and efficient process for the to prepare 4′-azido pyrimidine nucleosides.

In another embodiment of the present invention there is provided a method for the preparation of 4′-azidopurine derivatives of formula Ia wherein B is hypoxanthine, adenine or guanine wherein the 6-amino of adenine or the 2-amino of guanine is masked by an N-protecting group until the azido moiety is incorporated.

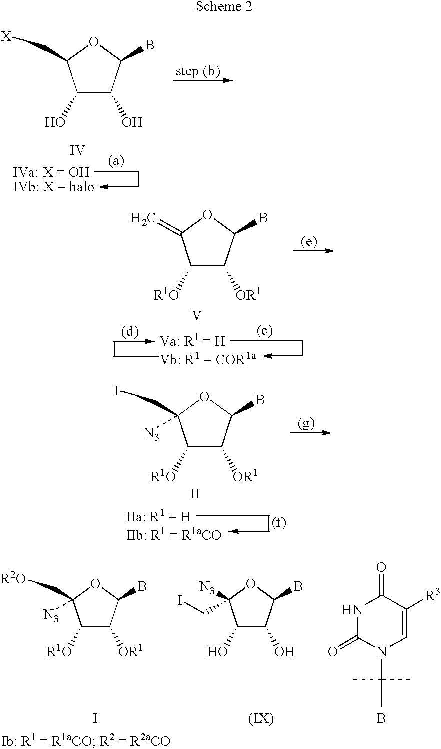

In another embodiment of the present invention there is provided a process (Scheme 2) for the preparation of a 4′- azido-2′,3′,5′-triacyl-nucleoside compound according to formula I, wherein R¹, R² and R³ are as defined hereinabove from a nucleoside IVa said process comprising (i) contacting IVa with a halogenating agent to afford IVb ; (ii) contacting IVb with a dehydrohalogenating agent to produce a 5-methylene nucleoside compound Va; (iii) contacting Va with a quaternary ammonium azide and iodine dissolved in a polar aprotic solvent to produce iodo azide IIa; (iv) contacting IIa with an acylating agent to afford diester IIb; and, (v) contacting a solution of a 5′-iodomethyl-2′,3′-diacyl nucleoside compound IIb in a suitable solvent with a carboxylic peracid, R^{2a}C(O)OOH, a carboxylic acid R^{2a}C(O)OH and optionally a phase transfer catalyst to afford I.

Elimination of protection/deprotection steps is generally desirable to minimize the number of discrete steps in a process. T. Tsuji and K. Takenaka (Nucleosides & Nucleotides 1987 6(3):575-80) disclose the bromination or iodination of the 5′-hydroxymethyl of unprotected nucleosides with carbon tetrahalides and TPP in DMF or HMPA. Maag et al. (supra) disclose a procedure for selective iodination of unprotected nucleosides by contacting a dioxane solution of the nucleoside with iodine and TPP in the presence of pyridine or imidazole. Yields of 29 to 59% were reported. Other methods for converting alcohols to iodides are well known (see, e.g., J. March, Advanced Organic Chemistry, John Wiley & Sons, 4^{th} edition, 1992, pp. 431-433). In the step (a) of the present process the reaction conditions have been improved and optimized by replacing dioxane with THF and carefully controlling the temperature of the reaction resulting in a yield of 84% of IVb.

Dehydrohalogenation of 5′-halonucleosides has been described (T. Ueda, in Chemistry of Nucleotides and Nucleosides, L. B. Townsend (ed.), Plenum Press, NY, 1988, pp. 83-88). Other examples of dehydrohalogenation reagents are described by March and references cited therein (J. March, supra pp. 1023-1025). Maag et al. (supra) disclosed the dehydroiodination of related nucleoside derivatives with DBN or sodium methoxide. In the step (b) of the present process dehydrogenation the sodium methoxide provided the best results. Excess sodium methoxide was quenched by addition of N-methylmorpholinium mesylate.

Step (e) of the present process is a formal electrophilic addition of iodine azide to the exocyclic olefin. In view of the known explosion and toxic hazards of organo azides (see, e.g., M.E.C. Biffin et al. in Chemistry of the Azido Group, S. Patai (ed.) Wiley-Interscience, New York, 1971, pp 61-63) and iodine azide (N. I. Sax and R. J. Lewis, Sr., Dangerous Properties of Industrial Materials, van Nostrand, New York 1989, p. 1993) synthetic reactions with organoazides must be carefully designed to minimize hazards as well as to increase efficiency. This is particularly true when the goal is a commercial scale manufacturing. Maag et al. (supra) and Moffatt (supra) both disclose the addition of iodine azide to 5-methylene nucleosides. Surprisingly it has now been found in differential scanning calorimetry and ARSST [Advanced Reaction System Screening Tool] that mixtures quaternary ammonium azides and iodine are less prone to violent decomposition then is iodine azide and efficiently add to 5-methylene nucleosides to afford iodo azides. Preferably the quaternary ammonium salt is a phase transfer catalyst and especially benzyl triethylammonium azide. Thus benzyl triethyl ammonium chloride and sodium azide are slurried in acetonitrile and the solution of benzyl triethylammonium azide (BTEAA) is filtered from the precipitated sodium chloride. A MeCN solution of the BTEAA, Va and NMM is cooled to 0-5° C. and a solution of iodine and THF was added to produce the desired iodo azide IIa and its diastereomer IX in a ratio of at least about 9:1 (IIa:IX). Excess azide is eliminated by addition of a small quantity of N-acetylcysteine which catalyzes oxidation of azide by iodine and the reaction product is then converted to the dibenzoate IIb (R^{1a}=Ph) in situ by addition of at least one base and benzoyl chloride while maintaining an internal temperature at ca. 5° C. The dibenzoate thus obtained is subjected to the percarboxylate/carboxylate (e.g., MCPBA/MCBA) mediated displacement to afford I.

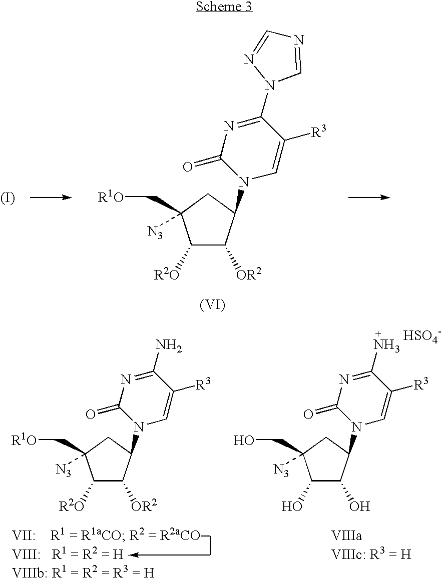

In another embodiment of the present invention there is provided a process (Scheme 2 & 3) for preparing 4′-azido-cytidine VIII wherein R³ is as defined hereinabove, said process comprising the steps (i) contacting nucleoside IVa with an halogenating agent to produce a 5′-halo nucleoside compound IVb; (ii) contacting IVb with a dehydrohalogenating agent to produce a 5-methylene nucleoside compound Va; (iii) contacting Va with a quaternary ammonium azide and iodine dissolved in an aprotic polar media to produce a iodo azide IIa; (iv) contacting IIa with an acylating agent to afford diester IIb wherein R¹, R² and R³ are as described hereinabove and (v) contacting a solution of a 5′-iodomethyl-2′,3′-diacyl nucleoside compound II in a suitable solvent with a carboxylic peracid, R^{2a}C(O)OOH, a carboxylic acid R^{2a}C(O)OH and optionally a phase transfer catalyst to afford I; (vi) contacting I with 1,2,4-triazole, phosphorus oxychloride and TEA in CH₂Cl₂ to afford triazole VI; (vii) contacting VI with a solution of ammonium hydroxide in an aprotic solvent (e.g., MeCN, THF or DMF) to displace the triazole to afford a 4′-azido-2′,3′,5′-triacylcytidine VII; (viii) contacting VII with a solution of ammonia and an alcohol to cleave the esters to afford VIII. The two step ammonolysis affords products of sufficient purity that no column chromatography is required to afford a final product with acceptable purity.

In another embodiment of the present invention there is provided a process (Scheme 2) for the preparation of a 4′-azido-2′,3′,5′-triacyl-nucleoside compound according to formula I, wherein R¹, R², and R³ are as defined hereinabove from nucleoside IVa said process comprising (i) contacting IVa with an halogenating agent to produce a 5′-halo nucleoside compound IVb wherein R¹ is hydrogen; (ii) contacting IVb with a dehydrohalogenating agent to produce a 5-methylene nucleoside compound Va; (iii) treating Va with an acylating agent to afford Vb, extracting a solution of Vb and a water immiscible solvent with aqueous NaHCO₃ with a pH at least about 7.5; (iv) removing the acyl groups to regenerate Va; (v) contacting Va with a quaternary ammonium azide and iodine dissolved in THF and MeCN to produce a iodo azide IIa; (vi) contacting IIa with an acylating agent to afford diester IIb; and (vii) contacting a solution of a 5′-iodomethyl-2′,3′-diacyl nucleoside compound II in a suitable solvent with a carboxylic peracid, R^{2a}C(O)OOH, a carboxylic acid R^{2a}C(O)OH and optionally a phase transfer catalyst to afford I; (viii) contacting I with 1,2,4-triazole, phosphorus oxychloride and TEA in CH₂Cl₂ to afford triazole VI; (ix) contacting VI with a solution of ammonium hydroxide and in a polar aprotic solvent to displace the triazole to afford a 4′-azido-2′,3′,5′-triacylcytidine VII; (x) contacting VII with a solution of ammonia and an alcohol to cleave the esters to afford VII.

In another embodiment of the present invention there is provided a process for preparing the hemisulfate acid addition salt of 4′-azidocytidine VIIIa said process comprising the preparation of VIII as described in Schemes 2 & 3 and further comprising the steps of adding sulfuric acid to the isopropanol/water mixture used to crystallize VIII.

Purification by column chromatography is a common laboratory technique but it is expensive and laborious in larger scale processes. In addition, chromatography produces large volume of solvents which are costly and have to be discarded or recycled. Thus modifications that eliminate chromatography are advantageous. Steps (iii) and (iv) in the previous embodiment can optionally be incorporated into the process to add an aqueous base wash if further purification of the 5-methylene nucleoside intermediate Va is required. Furthermore the dehydroiodination, acylation, washing and deacylation can be carried out in a single vessel.

In another embodiment of the present invention there is provided a process (Scheme 2 & 3) for preparing 4′-azido-cytidine VIIIa wherein R³ is hydrogen comprising the steps (i) contacting nucleoside IVa with TPP, iodine and imidazole to produce a 5′-iodo nucleoside compound IVb (X=I) wherein R¹ is hydrogen; (ii) contacting IVb (X═I) with DBN or sodium methoxide to produce a 5-methylene nucleoside compound Va; (iii) contacting Va with a benzyl triethylammonium azide and iodine dissolved in THF and MeCN to produce a iodo azide IIa; (iv) contacting IIa with benzoyl chloride to afford the diester IIb (R¹=PhCO); and (v) contacting a solution of a 5′-iodomethyl-2′,3′-diacyl nucleoside compound IIb (R¹=PhCO) in an aqueous buffer and a nonpolar organic solvent with a carboxylic peracid, R^{2a} C(O)OOH, a carboxylic acid R^{2a}C(O)OH and a phase transfer catalyst to afford Ib (R^{1a} is Ph and R^{2a} is optionally substituted Ph); (vi) contacting Ib (R^{1a} is Ph, R^{2a} is optionally substituted Ph and R³ is hydrogen) with 1,2,4-triazole, phosphorus oxychloride and TEA in CH₂Cl₂ to afford triazole VI (R^{1a} is Ph, R^{2a}is optionally substituted Ph and R³ is hydrogen); (vii) contacting VI (R^{1a} is Ph, R^{2a} is optionally substituted Ph and R³ is hydrogen) with a solution of ammonium hydroxide and acetonitrile to displace the triazole and afford a 4′azido-2′,3′,5′-triacylcytidine VII (R^{1a} is Ph, R^{2a} is optionally substituted Ph and R³ is hydrogen); (viii) contacting VII (R^{1a} is Ph, R^{2a}is optionally substituted Ph and R³ is hydrogen) with a solution of ammonium hydroxide and an alcohol to cleave the esters to afford VIIIb which crystallized from isopropanol/water/ sulfuric acid to afford the hemisulfate salt VIIIc.

Moffat (supra) disclosed the addition of iodine azide to 5′-methylene N-benzoyl cytidine (X). It has now been found that the over all process is more efficient when the nucleoside base is a 1H-pyrimidine-2,4-dione. The present embodiment is capable of producing uridine and cytidine nucleosides by inter-converting the bases linked to the nucleoside. In the present process there is provided a method to convert a uridine or thymine to a cytidine. The conversion of thymine to uridine by addition of triazoles has been described by Maag (supra), A. D. Borthwick (supra) and Divakar and Reese (supra). Displacement of the triazole with ammonia and cleavage of the triesters were best accomplished selectively by sequentially reacting VI (R¹ is R^{1a}CO and R² is R^{2a}CO and R³ is hydrogen) with ammonium hydroxide in a polar aprotic solvent which produced cytidine (VII) and reacting the triester VII with ammonia and methanol at about 45° C. which cleaved the benzoyl esters to afford VIII along with methyl benzoate and benzamide.

In another embodiment of the present invention there is provided a process (Scheme 2 & 3) for preparing the hemisulfate salt of 4′-azido-cytidine VIIIc wherein R¹ is PhCO, X is m-chloro-benzoyloxy, R² is m-chlorobenzoyl R^{2a} is m-chlorophenyl, and R³ is hydrogen said process comprising the steps (i) contacting nucleoside IVg with TPP, iodine and imidazole to produce a 5′-iodo nucleoside compound IVh; (ii) contacting IVh with a methanol solution of sodium methoxide to produce a 5-methylene nucleoside compound Vc; (iii) contacting Vc with a benzyl triethylammonium azide and iodine dissolved in THF and MeCN to produce a iodo azide IIe; (iv) contacting IIe with benzoyl chloride to afford diester IIg; (v) contacting a 5′-iodomethyl-2′,3′-diacyl nucleoside compound IIg with MCPBA, MCBA, tetrabutylammonium hemisulfate in a two-phase medium composed of DCM and aqueous potassium hydrogen phosphate (2 molar equivalents of K₂HPO₄ relative to MCPBA) to afford IIId; (vi) contacting IIId with 1,2,4-triazole, phosphorus oxychloride and TEA in CH₂Cl₂ to afford triazole IIIe; (vii) contacting IIIe with a solution of ammonium hydroxide and in a aprotic polar solvent to displace the triazole and afford a 4′azido-2′,3′,5′-triacylcytidine VII (R^{1a} is Ph, R^{2a} is 3-Cl-Ph, R³ is hydrogen); (viii) contacting VIII (R^{1a} is Ph, R^{2a}is 3-Cl-Ph, R³ is hydrogen) with a solution of ammonia and an methanol to cleave the esters to afford VIIb which crystallized from isopropanol/water/ sulfuric acid after removal of the MeOH to afford the hemisulfate salt VIIIc.

In another embodiment of the present invention there is provided novel compounds according to formula IX 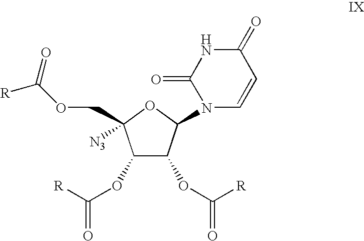
wherein R is phenyl optionally substituted with one to three substituents selected from the group consisting of halogen, C₁₋₁₀ alkyl, C₁₋₁₀ alkoxy, nitro, cyano which are useful intermediates in the preparation of VIIIc.

### Definitions

Unless otherwise stated, the following terms used in this Application, including the specification and claims, have the definitions given below. The phrase “a” or “an” entity as used herein refers to one or more of that entity; for example, a compound refers to one or more compounds or at least one compound. As such, the terms “a” (or “an”), “one or more”, and “at least one” can be used interchangeably herein.

In general, the systematic nomenclature used in this Application is based on AUTONOM™ v.4.0, a Beilstein Institute computerized system for the generation of IUPAC systematic nomenclature.

The phrase “as defined hereinabove” refers to the first definition provided in the Detailed Description of the Invention.

The term “alkyl” as used herein denotes an unbranched or branched chain, saturated, monovalent hydrocarbon residue containing 1 to 10 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkoxy group” as used herein means an -O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “C₁₋₁₀ alkoxy” as used herein refers to an -O-alkyl wherein alkyl is C₁₋₁₀.

The term “acyl” as used herein denotes a group of formula —C(═O)R wherein R is hydrogen, alkyl as defined herein or phenyl. The term or “alkylcarbonyl” as used herein denotes a group of formula C(═O)R wherein R is alkyl as defined herein. The term “arylcarbonyl” as used herein means a group of formula C(═O)R wherein R is an aryl group; the term “benzoyl” as used herein an “arylcarbonyl” group wherein R is phenyl.

The term “acylating agent” as used herein refers a compound capable of introducing an acyl group as defined above into a molecule. Acylating agents which react with hydroxyl, amine or sulfur substiuents commonly are either carboxylic acid anhydrides or acyl halides. The term “anhydride” as used herein refers to compounds of the general structure RC(O)—O—C(O)R wherein is as defined in the previous paragraph. The term “acyl halide” as used herein refers to the group RC(O)X wherein X is bromo or chloro. One skilled in the art will recognize that other “activated derivatives” of carboxylic acids are known in the art and these can also be used. The term “activated derivative” of a compound as used herein refers to a transient reactive form of the original compound which renders the compound reactive in a desired chemical reaction, in which the original compound is only moderately reactive or non-reactive.

The term “arylalkyl” or “aralkyl” as used herein denotes the radical R′R″-, wherein R′ is an aryl radical and R″ is an alkylene radical with the understanding that the attachment point of the arylalkyl moiety will be on the alkylene radical. The term “alkylene” as used herein denotes a divalent linear or branched saturated hydrocarbon radical, having from one to six carbons inclusive. The term aryl as used herein refers to a phenyl, a 1-naphthyl or a 2-napthyl moiety. Examples of alkylene radicals include, but are not limited to, methylene, ethylene, propylene, 2-methyl-propylene, butylene, 2-ethylbutylene. Examples of arylalkyl radicals include, but are not limited to, benzyl, phenylethyl and 3-phenylpropyl.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “halogen” or “halo” as used herein refers to fluorine, chlorine, bromine, or iodine.

The term “phase transfer catalyst” as used herein refers to a catalyst which alters the rate of transfer of water-soluble reactant across the interface to the organic phase where the anion or neutral compound can freely react with the organic reactant already located in the organic phase. Phase transfer catalysts commonly employed are quaternary ammonium salts, phosphonium salts and crown ethers or polyethylene glycols. Suitable catalysts are, e.g., tetrabutylammonium chloride, tetrabutylammonium bromide, tetrabutylphosphonium chloride, benzyl triethylammonium chloride, and N-2-ethylhexyl-4-dimethylamino pyridinium bromide. The term “quaternary ammonium azide” refers to a species R¹R²R³R⁴N⁺ N₃⁻ wherein R¹ to R⁴ are independently alkyl or aralkyl.

The phrase “polar aprotic solvent” as used herein denotes polar solvents which sufficiently polar to dissolve nucleoside derivatives but lack an exchangeable proton such as acetonitrile, DMF, dioxane, tetrahydrofuran, and the like.

The phrase “nonpolar organic solvent” as used herein means organic solvents such as, ligroin, pentane, hexane, cyclohexane, heptane, octane, benzene, toluene, diethyl ether, dioxane, tetrahydrofuran, dichloromethane, carbon tetrachloride, and the like.

The term “halogenating agent” as used herein refers to a reagent capable of converting an alcohol to an alkyl halide. The term “dehydrohalogenating agent” as used here refers to a compound capable of effecting the elimination of a hydrohalic acid HX from a haloalkane to afford an alkene.

The term “iodo azide” as used herein refers to two adjacent carbon atoms substituted by an iodide and by an azide, i.e. I—CH₂C(N₃)═.

The term “first base” refers to a base capable of reacting with an ester to afford the corresponding alcohol and carboxylic acid. Suitable first bases include ammonia, primary and secondary amines, NaHCO₃, Na₂CO₃, KOH and NaOMe and the like. The term “second base” refers to a base used in an acylation reaction as a catalyst and reagent to remove acid liberated during the transformation. Typical second bases include TEA, pyridine, DMAP, NMM, DABCO and the like.

The term “nucleoside” as used herein refers to a nitrogenous heterocyclic base linked to a pentose sugar by a glycosidic bond at C-1. Naturally occurring bases include uracil, thymine, cytosine, adenine and guanine and naturally occurring sugars are ribose and 2-deoxyribose. The term nucleoside further encompasses compounds in which the sugar and/or the nitrogenous base have been chemically modified.

As used herein, the term “treating”, “contacting” or “reacting” when referring to a chemical reaction means to add or mix two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

ABBREVIATIONS

| | |
|---|---|
| BTEAA | benzyl triethylammonium azide |
| DABCO | 1,4-diazabicyclo[2.2.2]octane |
| DBN | 1,8-diazabicyclo[4.3.0]non-5-ene |
| DCM | dichloromethane |
| DMF | N,N-dimethylformamide |
| DMAP | 4-dimethylaminopyridine |
| gc | gas chromatography |
| HMPA | hexamethylphosphoramide |
| hplc | high performance liquid chromatography |
| MCPBA | m-chloroperbenzoic acid |
| MCBA | m-chlorobenzoic acid |
| MeCN | acetonitrile |
| NMM | N-methylmorpholine |
| Ph | phenyl |
| TEA | triethylamine |
| THF | tetrahydrofuran |
| TPP | triphenylphosphine |

Although specific methods for producing 4′-azidonucleoside derivatives are described below, numerous modifications and alternative process steps will be apparent to those skilled in the art. Accordingly, this description and these examples are to be construed as illustrative only and is for teaching those skilled in the art novel processes for producing 4′-azidonucleoside derivatives. These processes may be varied substantially without departing from the spirit of the invention and the exclusive use of all modifications which come within the scope of the appended claim is reserved.

### EXAMPLE 1

### 1 -((2R,3R,4S,5 S)-3,4-Dihydroxy-5 -iodomethyl-tetrahydro-furan-2-yl)-1H-pyrimidine-2,4-dione (IVh)

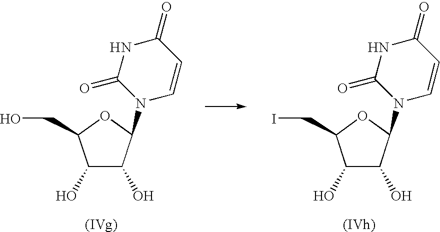

Uridine (IVg; 30.0 kg), TPP (46.8 kg) and imidazole (12.2 kg) were slurried in THF (267 kg). A solution of iodine (33.2 kg) in THF (87 kg) was added slowly to the slurry while the reaction temperature was maintained below 28° C. The reaction mixture was stirred overnight (ca. 18 h) at about 25° C. to achieve complete conversion. The reaction mixture was quenched with a small amount (2.3 L) of water. The reaction mixture was distilled under moderate vacuum while adding isopropanol (maximum internal temperature: 50° C.) till IPA content (by gc) of the distillate was greater than 87% (v/v). The resulting slurry was cooled to room temperature (ca. 22° C.) and aged overnight. The precipitated product was filtered and washed with isopropanol (2×50 kg) and dried at about 50° C. under vacuum with a slow nitrogen stream to afford IVh (36.5 kg; 83.9% theory.).

### EXAMPLE 2

### 1-((2R,3R,4S)-3,4-Dihydroxy-5-methylene-tetrahydro-furan-2-yl)-1H-pyrimidine-2,4-dione (Vc)

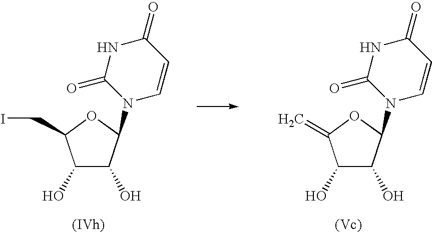

A suspension of IVh (4.0 kg) in MeOH (20 kg) was treated with 25% sodium methoxide solution (6.1 kg) to obtain a clear solution, which was maintained at ca 60° C. for about 2 h. The methanol was removed via vacuum distillation and replaced with MeCN until the methanol content (by gc) dropped to about 0.5% v/v. The volume of the resulting slurry was adjusted to about 30 L with acetonitrile and then treated with 3.5 kg of acetic anhydride and heated at about 60° C. for ca. 5 h. The reaction mixture was concentrated in vacuo to about 18 L and diluted with ethyl acetate. The reaction mixture was cooled to ca 20° C. and excess Ac₂O quenched by slow addition of a saturated NaHCO₃ until the pH of the solution was ca. 7.5. The phases were separated and the organic phase was washed with water and brine. The organic phase was concentrated in vacuo, diluted with MeOH (19 kg ) and treated with 1.08 kg of con. NH₄OH. The reaction mixture was allowed to stand overnight (ca. 18 h) at about 20° C. to complete the deacetylation. The reaction mixture was concentrated in vacuo (internal temp <40° C.) and the residue diluted with a mixture of isopropanol and acetonitrile. The final composition of solvent at the end of displacement distillation was typically: acetonitrile 65%, IPA 30%, methanol 5%. The olefin Vc precipitated and the resulting slurry was cooled to ca. 15° C., filtered, washed with cold acetonitrile and dried under in vacuo at 20-25° C. to yield Vc ( 1.92 kg; 75.1% theory).

### EXAMPLE 3

### Benzoic acid (2S,3 S,4R,5R)-4-benzoyloxyy-2-azido-5-(2,4-dioxo-3,4-dihydro-2H-pyrimidin-1-yl)-2-iodomethyl-tetrahydro-furan-3-yl ester (IIg)

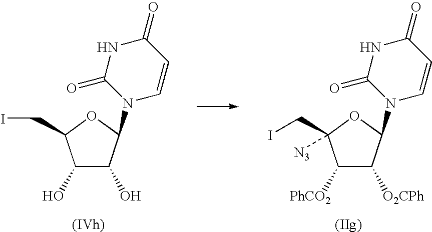

A suspension of IVh (12.0 kg) in MeOH (68 kg) was treated with 25% sodium methoxide solution (18.4 kg) to obtain a clear solution, which was allowed to stand at about 60° C. for about 2 h to achieve complete conversion. The reaction mixture was then added to a solution of N-methylmorpholinium mesylate in methanol (prepared in situ by adding 8.9 kg of NMM to a solution of 8.1 kg of methanesulfonic acid in 19 kg of MeOH). The reaction mixture was concentrated in vacuo (internal temp <40° C.) and the evaporated MeOH was replaced with THF (batch volume ca. 50 L) to until the residual methanol level was ca. 1-2% (by gc). The resulting slurry of crude Vc was diluted with acetonitrile (20 kg) and made slightly basic with NMM (1.2 kg). Benzyl triethylammonium chloride (10.0 kg) and sodium azide (2.87 kg) were slurried together in acetonitrile (45 kg) to extract azide into acetonitrile as the quaternary ammonium azide. The slurry was filtered, and the quaternary azide solution was added to the slurry of crude Vc. A solution of iodine (11.2 kg) in THF (40 kg) was then added slowly to the resulting slurry while maintaining batch temperature at 0-5° C. After completion of addition, the reaction mixture was allowed to stand at 5-10° C. for 18-24 hours to complete the conversion. To the reaction mixture was added TEA (17.2 kg) and DMAP (0.41 kg), and the mixture cooled to about −10° C. and treated with benzoyl chloride (14.3 kg) while maintaining the internal temperature below −5° C. After the addition was completed, the reaction mixture was allowed to stand at ca. −5° C. until benzoylation was complete. The reaction mixture was quenched with water and aqueous sodium sulfite (to destroy residual iodine) solution and treated with EtOAc (44 kg) was added. The organic phase was washed with water and water back-extracted with EtOAc (44 kg) and the combined organic extracts concentrated under reduced pressure (maximum jacket temperature: 65° C.) and the evaporated solvents were replaced with isopropanol from which IIg crystallized. The resulting slurry is cooled to ca. 20° C. and allowed to stand for at least 2 h. The precipitated product was isolated by filtration, washed with isopropanol and dried at 25-50° C. under a vacuum in a stream of nitrogen to yield IIg (15.9 kg; overall yield 77.6% theoretical)

### EXAMPLE 4

### Benzoic acid (2S,3S,4R,5R)-4-benzoyloxy-2-azido-5-(2,4-dioxo-3,4-dihydro-2H-pyrimidin-1-yl)-2-iodomethyl-tetrahydro-furan-3-yl ester (IIg)

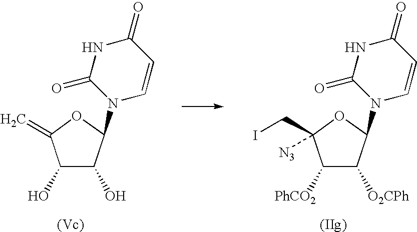 Step 1

A mixture of benzyl triethylammonium chloride (2.0 kg) and sodium azide (0.69 kg) was slurried together in MeCN (12 kg). The insoluble sodium chloride was removed by filtration and the filtrate washed with MeCN. To a homogenous mixture of Vc (1.9 kg), 4-NMM (0.26 kg) and THF (7.6 L) was added the MeCN solution of benzyl triethylammonium azide which produced a clear solution. A solution of iodine (2.45 kg) in THF (10 L) was added slowly while maintaining the internal temperature at 0-5° C. After the addition was completed the reaction mixture was aged at 5-10° C. for ca 2 h. Excess azide was destroyed by adding small amounts of N-acetyl cysteine (40 g) before proceeding.

Step 2

The crude reaction mixture from the previous step was treated with NMM (4.3 kg) and DMAP (100 g), cooled to about 0° C. and benzoyl chloride (2.6 kg) was added while maintaining internal temperature at ca. 5° C. After the addition was complete, the reaction mixture was stirred at ca. 5° C. for ca. 30 min. Water was carefully added to destroy excess benzoyl chloride, sodium sulfite was added to destroy residual iodine and EtOAc was added to extract desired product. The EtOAc solution was washed with water and concentrated in vacuo. The EtOAc thus removed was replaced by isopropanol (maximum jacket temperature: 65° C.) which resulted in crystallization of the desired product. The resulting slurry was cooled to ca. 22° C. and allowed to stand overnight. The precipitate filtered, washed with isopropanol and dried at room temperature in vacuo under a stream of nitrogen to yield IIg (3.80 kg; 74.2% theory)

### EXAMPLE 5

### 3-Chloro-benzoic acid (2R,3S,4R,5R) -2-azido-3,4-bis-benzoyloxy-5-(2,4-dioxo-3,4-dihydro-2H-pyrimidin-1-yl)-tetrahydro-furan-2-ylmethyl ester (IIId)

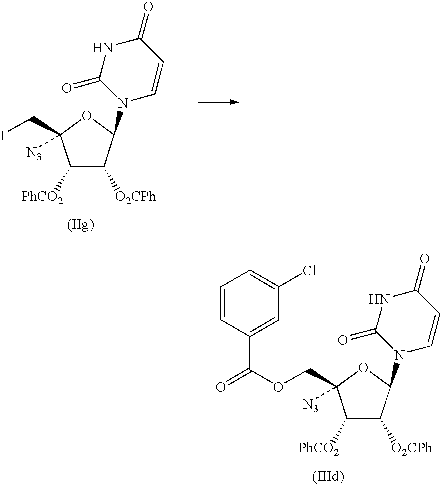

A mixture of IIg (14.2 kg), tetrabutyl ammonium hydrogen sulfate (8.5 kg), potassium hydrogen phosphate (8.5 kg), m-chlorobenzoic acid (4.0 kg), DCM (70 kg) and water (28 kg) was charged to a slurry of m-chloroperbenzoic acid (22.4 kg) in DCM (70 kg). The mixture was stirred at room temperature until the reaction was complete (by HPLC). To quench the reaction, the reaction mixture a solution of sodium sulfite (19 kg) in water (70 kg) was added while maintaining temperature below 25° C. After a stirring for a short time, a solution of potassium carbonate (28 kg) in water (51 kg) was added. The lower organic layer is separated and concentrated under atmospheric pressure. The DCM was replaced with isopropanol. The resulting solution (vol. 40-50 L) was treated with hot water (70 L) which resulted in the precipitation of the desired product. The resulting slurry was warmed to about 65° C. for 2 h and then allowed to cool to room temperature. The precipitated product was isolated by filtration, washed with a mixture of isopropanol and water and dried under vacuum at about 50° C. to afford IIId (10.6 kg; 71.3% theory)

### EXAMPLE 6

### 3-Chloro-benzoic acid (2R,3 S,4R5R) -2-azido-3,4-bis-benzoyloxy-5-(2-oxo-4-[1,2,4]triazol-1-yl-3,4-dihydro-2H-pyrimidin-1-yl)-tetrahydro-furan-2-ylmethyl ester (IIIe)

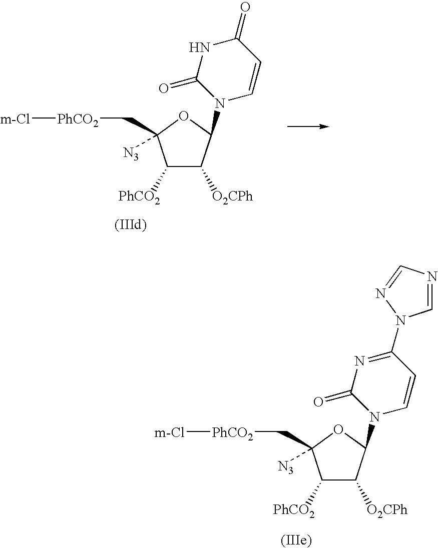

Phosphorous oxychloride (21.5 kg) was added to a cooled mixture of IIId (34.1 kg), 1,2,4-triazole (42.1 kg), TEA (63.1 kg) and DCM (270 kg) while the reaction temperature was maintained below 25° C. The reaction mixture was stirred at rt and the progress of the reaction monitored by hplc. When the reaction was complete the excess POCl₃ was quenched by careful addition of cold water (280 kg) while the reaction temperature was maintained below 30° C. The lower organic layer is separated and concentrated by distillation under atmospheric pressure. As the DCM distilled it was replaced by MeCN to maintain the volume approximately constant. The resulting slurry was (ca. 70 L) was diluted with water (70 L) which resulted in the precipitation of IIIe The resulting slurry was stirred at approximately 15° C. for up to 16 h. The precipitate was isolated by filtration, washed with a mixture of acetonitrile and water and dried under vacuum at about 50° C. to yield IIIe (32.7 kg; 88.8% theory).

### EXAMPLE 7

### 1-((2R,3 R,4S,5R)-5-Azido-3,4-dihydroxy-5-hydroxymethyl-tetrahydro-furan-2-yl)-2-oxo-1,2-dihydro-pyrimidin-4-yl-ammonium; hydrogen sulfate (VIIIc)

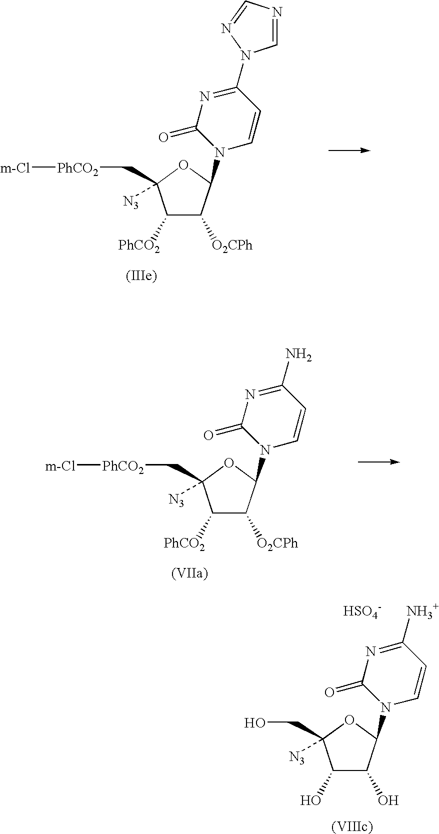

Triazole IIIe (32.2 kg) was suspended in THF (152 kg) and treated with conc. aqueous ammonium hydroxide (15 kg). After the ammonolysis reaction was completed, the reaction mixture was concentrated in vacuo and the methanol was added to bring the volume to ca. 180 L. To the resulting solution was added conc. aqueous ammonium hydroxide (15 kg) to cleave the protective ester functionality. After completion of the desired reaction, the solution was filtered, concentrated in vacuo and the reaction mixture was diluted with isopropanol to ca. 80 L. The resulting solution was diluted with isopropanol (64 kg) and water (for irrigation) (82 kg) which produced a clear solution. While warming up the solution to about 70° C., the solution was treated with dilute aqueous sulfuric acid -isopropanol mixture (prepared by mixing 2.4 kg of conc. sulfuric acid with 10 kg of water followed by addition of 68 kg of isopropanol). The resulting slurry is aged at ca. 70° C. for about 0.5 h and then cooled to ambient temperature over 2 hours. The precipitated product was isolated by filtration, washed with isopropanol and dried in vacuo with a nitrogen stream at about 50° C. to yield VIIIa (15.3 kg; 95.8% theory)

### Best Mode

The steps described in claim 8 represent the best mode of carrying out the process known to the inventors. These are described in Examples 1,3,5-7 respectively. These steps provide the most convenient and economical mode to practice the invention.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A process for the preparation of a 4′-azido-2′,3′,5′-triacyl-nucleoside compound according to formula I 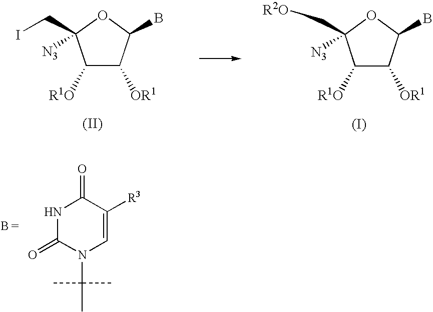
comprising contacting a solution of a 5′-iodo compound II in a suitable solvent with a peracid, R^{2a}C(O)OOH, an acid R^{2a}C(O)OH and optionally a phase transfer catalyst wherein R¹ is R^{1a}CO—, R² is R^{2a} CO— and R^{1a} and R^{2a}are independently C₁₋₁₀ alkyl or phenyl optionally substituted with 1 to 3 substituents selected from the group consisting of alkyl, alkoxy, halogen, nitro or cyano and R³ is selected from the group consisting of hydrogen, C₁₋₆ alkyl, C₁₋₃ haloalkyl and halogen.

2. A process as in claim 1 for the preparation of a 4′-azido-nucleoside compound according to formula Ia, said process further comprising contacting said 4′-azido-2′,3′,5′-triacyl-nucleoside compound I with a first base to afford a 4′-azido-nucleoside Ia.

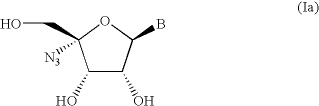

3. A process according to claim 1 said process further comprising the steps of:
(i) contacting nucleoside IVa with an halogenating agent to produce a 5′-halo nucleoside compound IVb wherein X is a halogen; 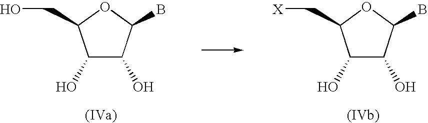
(ii) contacting IVb with a dehydrohalogenating agent to produce a 5-methylene nucleoside compound Va; 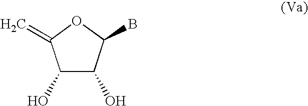
(iii) contacting (Va) with a quaternary ammonium azide and iodine dissolved in THF and MeCN to produce an iodo azide (IIa); 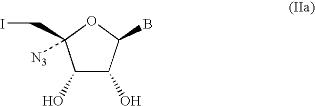
(iv) contacting IIa with at least one second base and a first acylating agent to afford diester II.

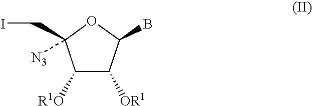

4. A process for preparing 4′-azidocytidine according to claim 3 said process further comprising the steps of:
(i) contacting I wherein R³ is hydrogen with 1,2,4-triazole, phosphorus oxychloride, TEA in CH₂Cl₂ to afford triazole VI; 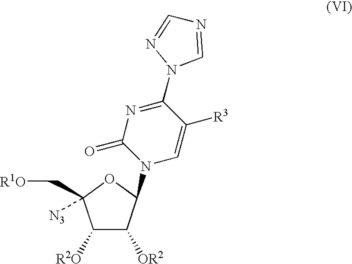
(ii) contacting VI with a solution of ammonium hydroxide and THF to displace the triazole to afford a 4′-azido-2′,3′,5′-triacylcytidine VII; 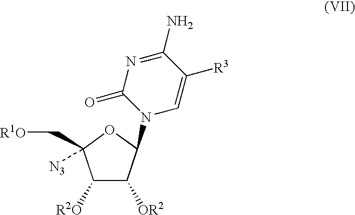
(iii) contacting VII with a solution of ammonia and an alcohol to cleave the esters to afford VIII.

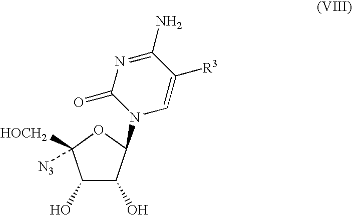

5. A process according to claim 4 said process further comprising the steps of:
(i) contacting Va with a second acylating agent and optionally with a trialkylamine base to afford diacyl compound Vb and washing an organic solution of Vb with aqueous NaHCO₃ wherein the pH was maintained at about 7.5; and, 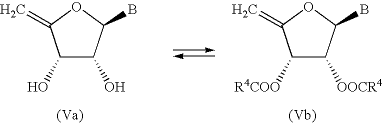
(ii) contacting said diacyl compound Vb with a solution of ammonia and methanol to regenerate Va.

6. A process according to claim 4 for preparing the hemisulfate acid addition salt of 4′-azidocytidine (VIIIa) said process further comprising the crystallizing VIII from isopropanol, water and sulfuric acid.

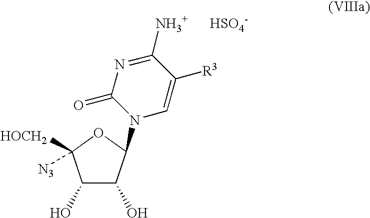

7. A process according to claim 6 for the preparation of the hemisulfate acid addition salt of 4′-azidocytidine VIIIa wherein R³ is hydrogen; R¹ is PhCO; R² is R^{2a}CO wherein R^{2a} is optionally substituted phenyl; said phase transfer catalyst is a tetraalkyl ammonium hydrogen sulfate, said solvent is mixture of an aqueous buffer and a nonpolar organic solvent, said iodinating agent is TPP, iodine and imidazole; said dehydrohalogenating agent is sodium methoxide or DBN; said quaternary ammonium azide is benzyl triethylammonium azide and said acylating agent is benzoyl chloride, said first base is ammonia and said second base is a NMM and DMAP and said alcohol is MeOH.

8. A process for the preparation of 4′-azidocytidine hemisulfate (VIIIc) 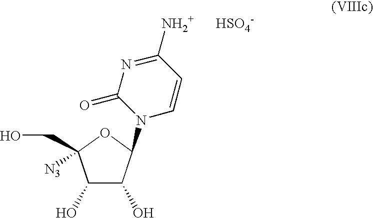
comprising the steps of:
(i) contacting a THF solution uridine (IVg) with TPP, iodine and imidazole to produce 1-(3,4-dihydroxy-5-halomethyl-tetrahydro-furan-2-yl)1H-pyrimidine-2,4-dione (IVh); 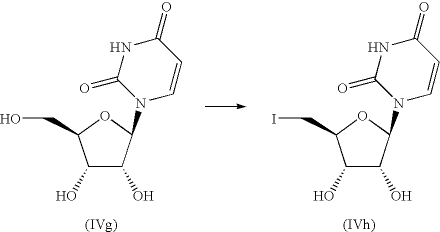
(ii) contacting IVh with a methanolic solution of sodium methoxide agent to produce 1-(3,4-dihydroxy-5-methylene-tetrahydro-furan-2-yl)1H-pyrimidine-2,4-dione (Vb); 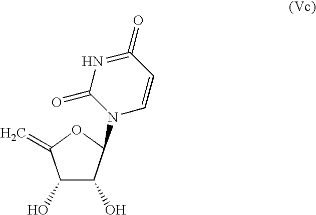
(iii) contacting Vc with a solution of benzyl triethylammonium azide and iodine in THF and MeCN to afford iodo azide IIe; 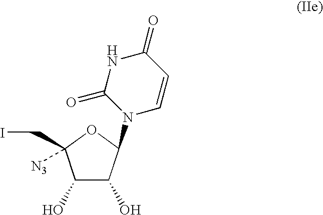
(iv) contacting IIe with a solution benzoyl chloride, NMM and MeCN to afford the dibenzoate IIg: 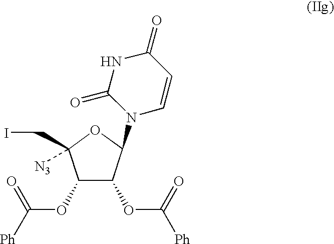
(v) contacting IIg with m-chloroperbenzoic acid, m-chlorobenzoic acid and tetrabutylammonium hydrogen sulfate in a two-phase solution of DCM and an aqueous solution of potassium hydrogen phosphate to afford compound IIId; 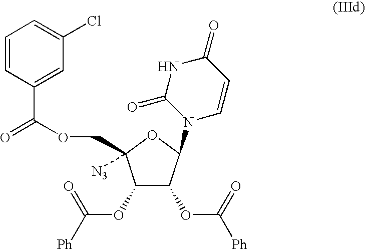
(vi) contacting IIId with a solution of 1,2,4-triazole, phosphorus oxychloride, TEA in DCM to afford IIIe; 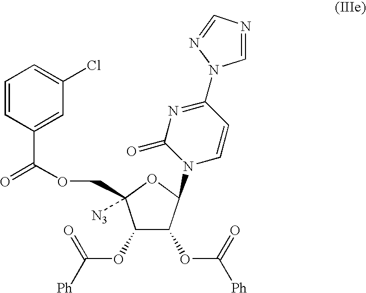
(vii) contacting IIIe with methanol solution of ammonia to cleave the esters and afford free base VIIIb. 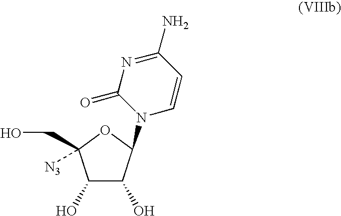
(viii) crystallize VIIIb from isopropanol and water containing H₂SO₄ to afford the hemisulfate salt VIIIc.

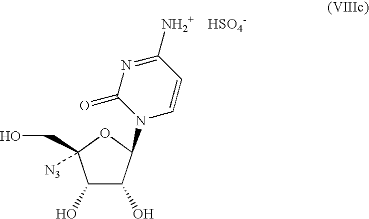

9. A compound according to formula IX 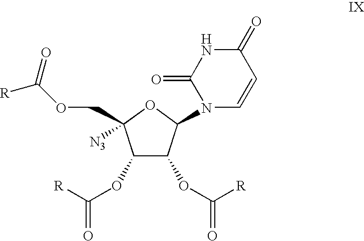
wherein R is phenyl optionally substituted with one to three substituents selected from the group consisting of halogen, C₁₋₁₀ alkyl, C₁₋₁₀ alkoxy, nitro, cyano.
